(19) United States
(12) Patent Application Publication
(10) Pub. No.: US 2023/0183157 A1
(43) Pub. Date: Jun. 15, 2023

(54) PROCESSES FOR THE PREPARATION OF HALOGENATED DIHYDROXYBENZENE COMPOUNDS

(71) Applicant: PURISYS LLC, ATHENS, GA (US)

(72) Inventors: Daniel M. HALLOW, Bishop, GA (US); Mark C. DOBISH, Watkinsville, GA (US); Gnel MKRTCHYAN, Watkinsville, GA (US); Richard WOOD, West Sacrament, CA (US); Ramanaiah C. KANAMARLAPUDI, Hockessin, DE (US); Achintya SUJAN, Athens, GA (US); Amber BURCH, Wilmington, DE (US)

(21) Appl. No.: 17/614,870

(22) PCT Filed: May 29, 2020
(86) PCT No.: PCT/US2020/035351

§ 371 Date: Nov. 29, 2021

Related U.S. Application Data
(60) Provisional application No. 62/855,072, filed on May 31, 2019.

(51) Int. Cl. C07C 37/62; C07C 39/24

(52) U.S. Cl. CPC C07C 37/62; C07C 39/245

(57) ABSTRACT

The present disclosure relates to the preparation of halogenated dihydroxybenzene compounds with high yield, selectivity and purity. The compounds are useful, among other things, in the synthesis of cannabinoids and cannabinoid-type compounds.

### CROSS-REFERENCE TO RELATED APPLICATIONS

This application is a national stage entry of International Application No. PCT/US2020/035351 filed May 29, 2020, which claims the benefit of U.S. provisional Application No. 62/855,072 filed May 31, 2019, which are hereby incorporated by reference.

### FIELD

The subject matter described herein relates to the preparation of halogenated dihydroxybenzenes by novel synthetic routes to improve purity and yield, and to lower costs and environmental impact in the preparation of these useful compounds.

### BACKGROUND

Dihydroxybenzene compounds are among the compounds known as phenols. These compounds are ubiquitous in nature and in modern chemicals. Many are useful in their own right and also as building blocks for other compounds. For example, one such compound is olivetol. Olivetol (also known as 5-pentylresorcinol or 5-pentyl-1,3-benzenediol, 5-n-amylresorcinol, and 3,5-dihydroxyamylbenzene) is a naturally occurring phenolic-type compound.

However useful these compounds, there is always a need for improved syntheses that utilize these compounds. As such, derivatives of dihydroxybenzenes that introduce new chemical handles are among the most useful types of compounds. However, the presence of the hydroxy groups on the benzene ring can lead to unwanted side reactions, requiring blocking groups.

Additionally, the reported syntheses for some desirable derivatives can require expensive and/or toxic reagents and byproducts. As such, handling of the reactions and the by-products they produce can be cost prohibitive. Further, it is desired to mitigate the environmental toll that many of these reactions produce.

What is therefore needed are efficient, scalable, low-cost, low environmental impact synthetic methods for preparing halogenated alkyl-substituted dihydroxybenzenes. The subject matter described herein addresses these unmet needs.

### BRIEF SUMMARY

In certain aspects, the subject matter described herein is directed to methods of preparing a compound of Formula I:

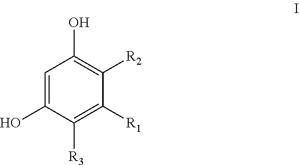

wherein,

R₁ is a branched or straight chain C₁₋₁₂ alkyl; and

R₂ and R₃ are each independently selected from the group consisting of halogen, —C(O)O—C₁₋₆alkyl, and hydrogen, wherein at least one of R₂ and R₃ is halogen, the methods comprising:

contacting a compound of Formula I′ having a structure:

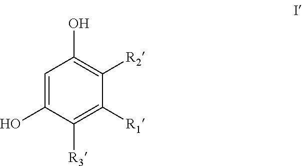

with HX, wherein X is a halide, in the presence of an organic sulfoxide

wherein, R_{1′} is a branched or straight chain C₁₋₁₂ alkyl; and

R_{2′} and R_{3′} are each independently selected from the group consisting of hydrogen, —C(O)O—C₁₋₆alkyl and halogen, wherein at least one of R_{2′} and R_{3′} is hydrogen;

wherein, the contacting is at a temperature from about 0° C. to about 100° C.; and

wherein, a compound of Formula I is prepared.

In certain aspects, the subject matter described herein is directed to methods of preparing a compound of Formula I:

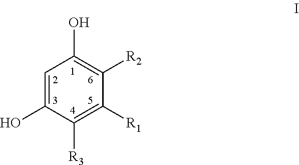

wherein,

R₁ is a branched or straight chain C₁₋₁₂alkyl; and

R₂ and R₃ are each halogen,

the methods comprising:

selectively halogenating at the 4- and 6-positions by contacting a compound of Formula I′ having a structure:

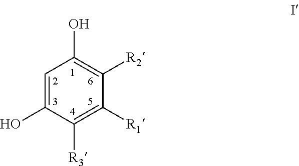

with a first solvent to form a mixture,

contacting the mixture with HX, wherein X is a halide, in the presence of an organic sulfoxide;

wherein, R_{1′} is a branched or straight chain C₁₋₁₂alkyl; and

R_{2′} and R_{3′} are each hydrogen;

wherein, the contacting is at a temperature from about 0° C. to about 100° C.; and

wherein, a compound of Formula I is prepared.

These and other aspects are described fully herein.

### BRIEF DESCRIPTION OF THE FIGURES

FIG. 1 shows the structures of several compounds produced in a process for preparing 4,6-dibromo-olivetol (“DBO”).

FIG. 2 depicts a HPLC trace of DBO, showing the levels of certain impurities. (HPLC method: Column: waters XBridge Shield RP18 3.5 μm, 3.0×150 mm, PN:186003041; column temp 35° C.; MPA: 0.05% (v/v) Acetic acid in water/Acetonitrile 95/5 (v/v); MPB: Methanol, UV wave length 225 nm; Flow rate 0.7 mL·min. MP Gradient: 0 min MPA 40%, 19 min MPA 5%, 21 min MPA 5%, 21.1 min MPA 40%, 25 min MPA 40%.)

FIGS. 3A & 3B depict data that indicate that temperature and reactive species have a predominant effect on the formation of 4,6-DBO (product) measured at the end of 6 hours under reaction conditions.

FIGS. 4A & 4B depict data that indicate reactive species and ethyl acetate volume have a predominant effect on the formation of 4-MBO measured at the end of 6 hours under reaction conditions.

FIGS. 5A & 5B depict data that indicate reactive species and ethyl acetate volume have a predominant effect on the formation of 2,4-DBO measured at the end of 6 hours under reaction conditions.

FIGS. 6A & 6B depict data that indicate that temperature, reactive species and ethyl acetate volume have a predominant effect on the formation of 2,4,6-TBO measured at the end of 6 hour under reaction conditions.

FIG. 7 depicts a HPLC trace of DBO, showing the levels of certain impurities. (HPLC method: Column: waters XBridge Shield RP18 3.5 μm, 3.0×150 mm, PN:186003041; column temp 35° C.; MPA: 0.05% (v/v) Acetic acid in water/Acetonitrile 95/5 (v/v); MPB: Methanol, UV wave length 225 nm; Flow rate 0.7 mL·min. MP Gradient: 0 min MPA 40%, 19 min MPA 5%, 21 min MPA 5%, 21.1 min MPA 40%, 25 min MPA 40%.)

FIG. 8 depicts a GC analysis of the product of Example 13.

### DETAILED DESCRIPTION

Disclosed herein are efficient synthetic routes to produce halogenated dihydroxybenzenes with improved purity, efficiency, and safety, and with lower volumes, lower energy costs and lower environmental impact. It has now been found that desired halogenated dihydroxybenzenes can be prepared using relatively mild conditions, without the need for specialized handling and equipment associated with the use of corrosive materials, such as diatomic bromine (Br₂), without the need for cryogenic conditions and the equipment needed for such conditions, and/or in the absence of toxic solvents, such as dichloromethane. Also important for large scale manufacturing is the need for only one vessel for the entire reaction, whereas known methods for producing halogenated olivetol can require two vessels.

In particular, known synthetic routes to produce halogenated olivetol, such as a dibromo-olivetol (DBO) generally involve reacting olivetol and diatomic bromine in a solvent, such as dichloromethane, at −15° C. See Scheme 1.

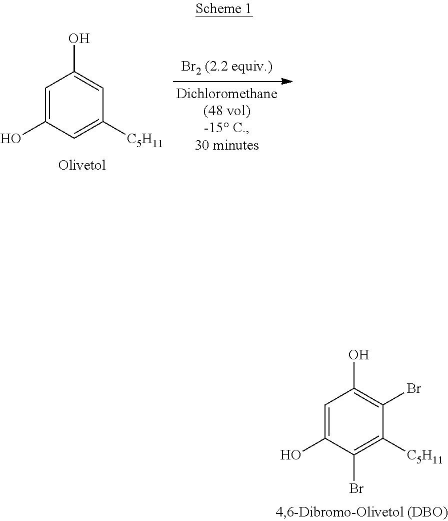

Diatomic bromine is acutely toxic, highly corrosive and needs special precautions to handle. Also, the reaction requires low temperature (−15° C.), is unstable, often results in bromine scrambling, requires high volume (Vₘₐₓ 48) or higher, and precise dosing, which is often beyond the capabilities of existing standard equipment. To overcome these challenges for producing, in particular, DBO, and to reduce manufacturing costs and environmental impacts, novel processes as described herein have been developed (See Schemes 2, 3 and 4). Other methods utilizing bromodimethylsulfonium have been shown to produce halogenated arene species. (Majetich G., et al. JOC, 62, 4321-4326 (1997); Song S., et al., Org. Lett. 17, 2886-2889 (2015)). However, notably, of the numerous scaffolds disclosed, none are alkylated dihydroxybenzenes.

The presently disclosed reaction conditions use aqueous HX as the halide source in an appropriate co-solvent/oxidation system, such as DMSO and ethyl acetate. The materials and reaction conditions are less corrosive and safer to handle. Other advantages involve an increase in the reaction and workup temperatures, such that there is no requirement for sub-zero temperatures. Further advantages include the removal of the known carcinogen dichloromethane from the reaction. Advantageously, the overall yield and impurity profile improved substantially. Notably, unlike other reactions, there is tunable control on the Formula I scaffold that provides selective halogenation. Another advantage is the substantially lower amount or absence of bromine scrambling that has been observed with the reaction of the type depicted in Scheme 1.

It was not known whether the process could selectively halogenate the scaffold of formula I in a desired manner. The numbered positions are as shown below:

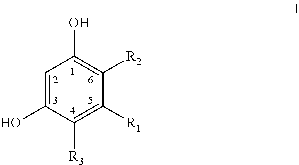

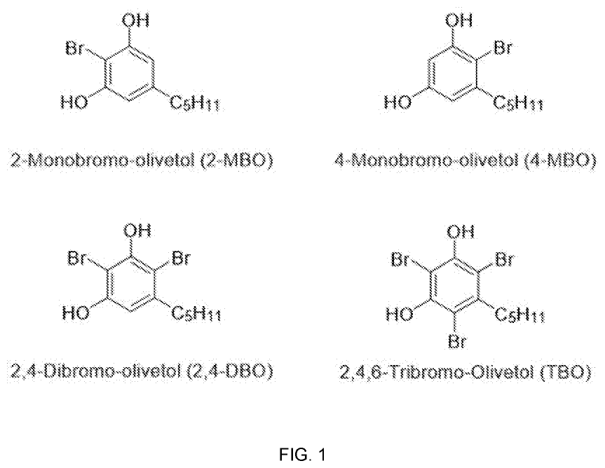

In certain embodiments, the desired products of the methods described herein are the 4,6-dihalogenated compounds. The methods described herein have been shown to yield the desired products at high ratios relative to other halogenated impurities. The structures of certain impurities are; 4-monobromo-olivetol (4-MBO); 2,4-dibromo-olivetol (2,4-DBO); and 2,4,6-tribromo-olivetol (TBO). Another impurity that can be present is 2-MBO. Thus, in certain embodiments, the 2-position of Formula I can be substituted with a halogen, as a minor byproduct, for example, 20% or less relative formation, of the syntheses. The structures of several of these compounds are shown in FIG. 1.

Advantageously, the methods described herein provide for large-scale preparation of the desired compounds of Formula I at high purity, e.g., >99A %, and at excellent yields, e.g., in certain embodiments, above about 90% without the need to use corrosive diatomic bromide and toxic dichloromethane.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Definitions

As used herein, the term “alkyl” refers to an unbranched or branched saturated hydrocarbon chain. As used herein, alkyl has 1 to 12 carbon atoms (i.e., C₁-C₁₂ alkyl), 1 to 8 carbon atoms (i.e., C₁-C₈ alkyl), 1 to 6 carbon atoms (i.e., C₁-C₆ alkyl), 1 to 5 carbon atoms (i.e., C₁-C₅ alkyl), or 3 to 5 carbon atoms (i.e., C₃-C₅ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

As used herein, the terms “halogen,” or “halo” refer to atoms occupying group VIIA of the periodic table, such as fluoro, chloro, bromo or iodo. The term “halide” refers to the halogen or halo in a binary compound with hydrogen.

As used herein, C₁₋₆ alkyl esters can be depicted as “—C(O)O—C₁₋₆ alkyl” where the moiety is attached to the phenyl ring at the carbonyl.

As used herein, the term “contacting” refers to allowing two or more reagents to contact each other. The contact may or may not be facilitated by mixing, agitating, stirring, and the like.

As used herein, the term “selectively halogenating” refers to the halogenation at specific position of the aryl ring, such as the 4′ and 6-positions, such that the yield and purity of the 4-, 6-dihalogenated compound of Formula I has the desired yield and purity as disclosed elsewhere herein.

In the structures shown herein, where the stereochemistry of any particular chiral atom is not specified, then all stereoisomers are contemplated and included as the compounds of the invention.

Additional definitions may be provided herein.

### II. Synthetic Methods

In an aspect, the subject matter described herein is directed to methods of preparing a compound of Formula I:

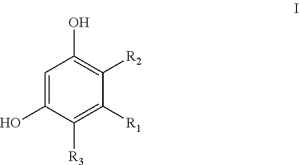

wherein:

R₁ is a branched or straight chain C₁₋₁₂ alkyl; and

R₂ and R₃ are independently selected from the group consisting of halogen, —C(O)O—C₁₋₆alkyl, and hydrogen, wherein at least one of R₂ and R₃ is halogen; the methods comprising:

contacting a compound of Formula I′ having a structure:

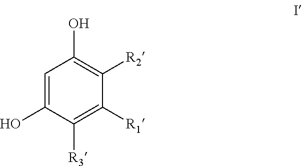

wherein, R_{1′} is a branched or straight chain C₁₋₁₂ alkyl; and

R_{2′} and R_{3′} are each independently selected from the group consisting of hydrogen, —C(O)O—C₁₋₆alkyl, and halogen, wherein at least one of R_{2′} and R_{3′} is hydrogen;

with HX, wherein X is a halide, in the presence of an organic

wherein the contacting is at a temperature from about 0° C. to about 100° C.; and

wherein the compound of Formula I is prepared.

In certain embodiments, the halogen is selected from the group consisting of Br, Cl, F, and I. In certain embodiments, the halogen is selected from the group consisting of Br and Cl. In certain embodiments, the halogen is Br.

In certain embodiments, the HX is selected from the group consisting of HBr, HCl, HF, and HI. In certain embodiments, the HX is selected from the group consisting of HBr and HCl. In certain embodiments, the HX is HBr. In certain embodiments, the HX is an aqueous solution. In certain embodiments, the HX is a 10% to 90% aqueous solution. In certain embodiments, the HX is a 20% to 80% aqueous solution. In certain embodiments, the HX is a 30% to 70% aqueous solution. In certain embodiments, the HX is a 40% to 60% aqueous solution. In certain embodiments, the HX is a 45% to 55% aqueous solution. In certain embodiments, the HX is a 46% to 50% aqueous solution, such as, aq. 48% HBr.

In certain embodiments, HX is present in an amount of about 1.5-3.0 molar equivalents. In certain embodiments, HX is present in an amount of about 2.0-3.0 molar equivalents. In certain embodiments, HX is present in an amount of about 1.8-2.5 molar equivalents. In certain embodiments, HX is present in an amount of about 2.0-2.3 molar equivalents. In certain embodiments, HX is present in an amount of about 1.9 molar equivalents, 2.0 molar equivalents, 2.1 molar equivalents, 2.2 molar equivalents, or 2.3 molar equivalents.

In certain embodiments, R₁ is a straight or branched or straight chain C₁₋₁₂ alkyl selected from the group consisting of methyl, ethyl, n-propyl, isopropyl, n-butyl, sec-butyl, isobutyl, t-butyl, pentyl, hexyl, heptyl, octyl, nonyl, decyl, undecyl, and dodecyl (including any isomers of each). In certain embodiments, R₁ is a branched or straight chain C₁₋₁₂ alkyl selected from the group consisting of methyl, ethyl, propyl, pentyl, and hexyl (including isomers of each). In certain embodiments, R₁ is a straight chain C₁₋₁₂ alkyl selected from the group consisting of propyl and pentyl. In certain embodiments, R₁ is a branched chain C₁₋₁₂ alkyl having one, two, three, four, five, six, seven, eight, nine, ten, eleven, or twelve carbon atoms.

In certain embodiments, R_{1′} is a straight or branched or straight chain C₁₋₁₂ alkyl selected from the group consisting of methyl, ethyl, n-propyl, isopropyl, n-butyl, sec-butyl, isobutyl, t-butyl, pentyl, hexyl, heptyl, octyl, nonyl, decyl, undecyl, and dodecyl (including any isomers of each). In certain embodiments, R_{1′} is a branched or straight chain C₁₋₁₂ alkyl selected from the group consisting of methyl, ethyl, propyl, pentyl, and hexyl (including isomers of each). In certain embodiments, R_{1′} is a straight chain C₁₋₁₂ alkyl selected from the group consisting of propyl and pentyl. In certain embodiments, R_{1′} is a branched chain C₁₋₁₂ alkyl having one, two, three, four, five, six, seven, eight, nine, ten, eleven, or twelve carbon atoms. Those of skill in this field would recognize the corresponding nature of R and R′ groups in the starting materials and products of the reactions.

In all embodiments, at least one of R_{2′} and R_{3′} is hydrogen. The other of R_{2′} and R_{3′} is selected from the group consisting of hydrogen, an acid ester such as —C(O)O—C₁₋₆alkyl, and halogen. In certain embodiments, the halogen is bromine. In certain embodiments, in the —C(O)O—C₁₋₆alkyl, the C₁₋₆alkyl is methyl, ethyl, propyl or butyl. In certain embodiments, the C₁₋₆alkyl is methyl, i.e., —C(O)O-Me, or ethyl, i.e., —C(O)O-Et.

In certain embodiments, the compound of Formula I′ is dissolved in a solvent prior to contacting the compound of Formula I′ with HX in the presence of an organic sulfoxide, such as DMSO. In certain embodiments, the solvent is selected from the group consisting of ethyl acetate, isopropyl acetate, acetonitrile, acetone, t-butyl methyl ether, ethanol, dichloromethane, n-heptane, toluene, 2-Me-THF, and isopropanol. In certain embodiments, the solvent is selected from the group consisting of ethyl acetate, isopropyl acetate, acetonitrile and acetone. In certain embodiments, the solvent is ethyl acetate.

The volume of solvent can be adjusted. As described elsewhere herein, the amount of solvent can impact the formation of the desired compound(s) of Formula I. In certain embodiments, the amount of solvent can be about 5 volumes, 6 volumes, 7 volumes, 8 volumes, 9 volumes, 10 volumes, 11 volumes, 12 volumes, 13 volumes, 14 volumes, 15 volumes, 16 volumes, 17 volumes, 18 volumes, 19 volumes, 20 volumes, 21 volumes, 22 volumes, 23 volumes, 24 volumes, 25 volumes, or more. In certain embodiments, the solvent is present in a range from about 5 volumes to about 25 volumes; or about 9 volumes to about 17 volumes; or about 9.7 volumes to about 16.1 volumes. In certain embodiments, the solvent is ethyl acetate in an amount of at least 15 volumes.

Without being bound to theory, the organic sulfoxide, such as DMSO, may act as an oxidant. When performing the selective halogenation, the amount of organic sulfoxide, such as DMSO, can vary. In certain embodiments, organic sulfoxide, such as DMSO is present in an amount of about 1.5-5.0 molar equivalents. In certain embodiments, organic sulfoxide, such as DMSO is present in an amount of about 1.8-4.5 molar equivalents. In certain embodiments, organic sulfoxide, such as DMSO is present in an amount of about 2.0-3.0 molar equivalents. In certain embodiments, organic sulfoxide, such as DMSO is present in an amount of about 1.9 molar equivalents, 2.0 molar equivalents, 2.1 molar equivalents, or 2.2 molar equivalents. The organic sulfoxide can be those known in the art and ave the formula:

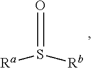

where, R^{a} and R^{b}, are each independently benzyl, phenyl, alkyl, aryl or allyl. In certain embodiments, the organic sulfoxide is a di-C₁₋₆ alkyl sulfoxide, such as DMSO. Other exemplary organic sulfoxides include those where:

| R^{a} | R^{b} |
|---|---|
| Methyl | Methyl |
| Methyl | Phenyl |
| Benzyl | Benzyl |
| Phenyl | Phenyl |
| Methyl | Benzyl |
| Allyl | Allyl |
| Benzyl | Phenyl |
| Ethyl | Ethyl |
| Propyl | Propyl |
| Butyl | Butyl |
| Benzyl | Cyclohexyl |

In certain embodiments, the contacting is at a temperature of from about 0° C. to about 100° C.; or from about 10° C. to about 90° C.; or from about 20° C. to about 80° C.; or from about 30° C. to about 70° C.; or from about 40° C. to about 60° C.; or from about 45° C. to about 55° C. In certain embodiments, the contacting is at a temperature from about 0° C. to about 20° C., from about 20° C. to about 25° C., from about 25° C. to about 30° C., from about 30° C. to about 35° C., from about 35° C. to about 40° C., from about 40° C. to about 45° C., from about 45° C. to about 50° C., from about 50° C. to about 55° C., from about 55° C. to about 60° C., from about 60° C. to about 65° C., from about 65° C. to about 70° C., about 70° C. to about 75° C., from about 75° C. to about 80° C., or from about 80° C. to about 100° C. In certain embodiments, the methods do not include any cooling.

In certain embodiments, the contacting is for a period of time from about 5 minutes to about 24 hours, or from about 30 minutes to about 20 hours, or from about 30 minutes to about 15 hours, or from about 30 minutes to about 10 hours, or from about 30 minutes to about 5 hours, or from about 30 minutes to about 3.5 hours, or from about 1 hour to about 3 hours, or from about 1.5 hours to about 2.5 hours. In certain embodiments, the contacting is for a period of time of about 5 minutes, about 10 minutes, about 20 minutes, about 30 minutes, about 45 minutes, about 1 hour, about 1.25 hours, about 1.5 hours, about 1.75 hours, about 2 hours, about 2.25 hours, about 2.5 hours, about 2.75 hours, about 3 hours, about 3.25 hours, about 3.5 hours, about 3.75 hours, about 4 hours, about 4.25 hours, about 4.5 hours, about 4.75 hours, about 5 hours, about 10 hours, about 15 hours, about 20 hours, about 24 hours, or more.

In certain embodiments, the method produces the compound of Formula I having a purity above about 88A %, above about 90A %, above about 92A %, above about 93A %, above about 94A %, above about 95A %, above about 96A %, above about 97A %, above about 98% or above about 99A %.

In certain embodiments, the compound of Formula I has one of the following structures, where R₂ and R₃ are independently selected from the group consisting of halogen, —C(O)O—C₁₋₆alkyl, and hydrogen, wherein at least one of R₂ and R₃ is halogen:

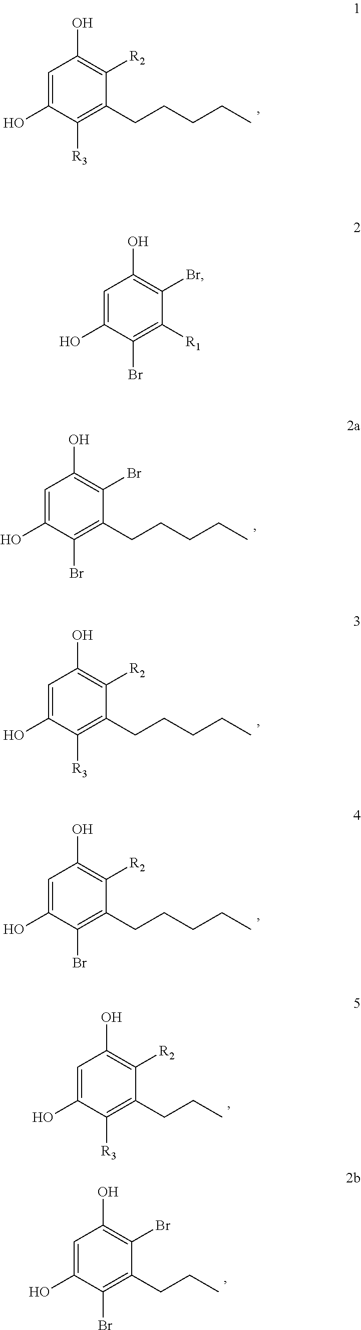

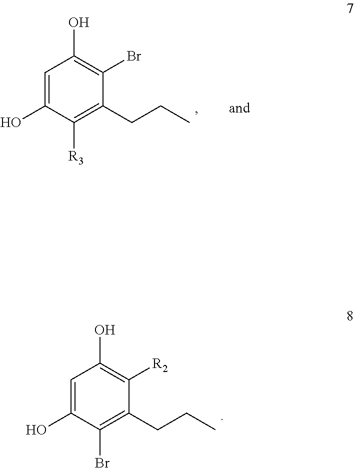

In certain embodiments, the compound of Formula I is compound 2a. In certain embodiments, the compound of Formula I is compound 2b.

In another aspect, the subject matter is directed to methods of preparing a compound of Formula I:

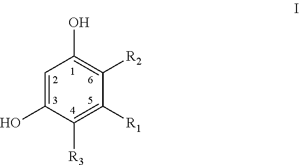

wherein,

R₁ is a branched or straight chain C₁₋₁₂ alkyl; and

R₂ and R₃ are each halogen,

the methods comprising:

selectively halogenating at the 4- and 6-positions by contacting a compound of Formula I′ having a structure:

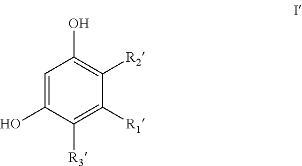

wherein, R_{1′} is a branched or straight chain C₁₋₁₂ alkyl; and

R_{2′} and R_{3′} are each hydrogen; and

with a first solvent to form a mixture,

contacting the mixture with HX, wherein X is a halide, in the presence of an organic sulfoxide;

wherein, the contacting the mixture with HX is at a temperature from about 0° C. to about 100° C.; and

wherein, the compound of Formula I is prepared.

In certain embodiments, the method of selectively halogenating produces the compound of Formula I, which is present at a ratio of at least 10:1 relative to certain impurities, such as: mono-halogenated, tri-halogenated or 2,4-dihalogenated compounds. In certain embodiments, the ratio is at least 11:1; at least 12:1; at least 13:1; at least 14:1; at least 15:1; at least 16:1; at least 17:1; at least 18:1; at least 19:1; at least 20:1; at least 21:1; at least 22:1; at least 23:1; at least 24:1; at least 25:1; at least 26:1; at least 27:1; at least 28:1; at least 29:1; at least 30:1; at least 31:1; at least 32:1; at least 33:1; at least 34:1; or at least 35:1. In certain embodiments, the ratio is from about 25:1 to about 35:1. In certain embodiments, a composition comprises 4,6-DBO; 4-MBO; 2,4-DBO and TBO in amounts of about 94%, about 3%, about 1%, and about 1%, respectively.

In certain embodiments of selectively halogenating, the halogen is selected from the group consisting of Br, Cl, F, and I. In certain embodiments, the halogen is selected from the group consisting of Br and Cl. In certain embodiments, the halogen is Br.

In certain embodiments of selectively halogenating, the HX is selected from the group consisting of HBr, HCl, HF, and HI. In certain embodiments, the HX is selected from the group consisting of HBr and HCl. In certain embodiments, the HX is HBr. In certain embodiments, the HX is an aqueous solution. In certain embodiments, the HX is a 10% to 90% aqueous solution. In certain embodiments, the HX is a 20% to 80% aqueous solution. In certain embodiments, the HX is a 30% to 70% aqueous solution. In certain embodiments, the HX is a 40% to 60% aqueous solution. In certain embodiments, the HX is a 45% to 55% aqueous solution. In certain embodiments, the HX is a 46% to 50% aqueous solution, such as, aq. 48% HBr.

In certain embodiments of selectively halogenating, HX is present in an amount of about 1.5-3.0 molar equivalents. In certain embodiments of selectively halogenating, HX is present in an amount of about 2.0-3.0 molar equivalents. In certain embodiments, HX is present in an amount of about 1.8-2.5 molar equivalents. In certain embodiments, HX is present in an amount of about 2.0-2.3 molar equivalents. In certain embodiments, HX is present in an amount of about 1.9 molar equivalents, 2.0 molar equivalents, 2.1 molar equivalents, 2.2 molar equivalents, or 2.3 molar equivalents.

In certain embodiments of selectively halogenating, R₁ is a straight or branched or straight chain C₁₋₁₂ alkyl selected from the group consisting of methyl, ethyl, n-propyl, isopropyl, n-butyl, sec-butyl, isobutyl, t-butyl, pentyl, hexyl, heptyl, octyl, nonyl, decyl, undecyl, and dodecyl (including any isomers of each). In certain embodiments, R₁ is a branched or straight chain C₁₋₁₂ alkyl selected from the group consisting of methyl, ethyl, propyl, pentyl, and hexyl (including isomers of each). In certain embodiments, R₁ is a straight chain C₁₋₁₂ alkyl selected from the group consisting of propyl and pentyl. In certain embodiments, R₁ is a branched chain C₁₋₁₂ alkyl having one, two, three, four, five, six, seven, eight, nine, ten, eleven, or twelve carbon atoms.

In certain embodiments of selectively halogenating, R_{1′} is a straight or branched or straight chain C₁₋₁₂ alkyl selected from the group consisting of methyl, ethyl, n-propyl, isopropyl, n-butyl, sec-butyl, isobutyl, t-butyl, pentyl, hexyl, heptyl, octyl, nonyl, decyl, undecyl, and dodecyl (including any isomers of each). In certain embodiments, R_{1′} is a branched or straight chain C₁₋₁₂ alkyl selected from the group consisting of methyl, ethyl, propyl, butyl, pentyl, and hexyl (including isomers of each). In certain embodiments, R_{1′} is a branched or straight chain C₁₋₁₂ alkyl selected from the group consisting of propyl and pentyl. In certain embodiments, R_{1′} is a branched chain C₁₋₁₂ alkyl having one, two, three, four, five, six, seven, eight, nine, ten, eleven, or twelve carbon atoms.

In all embodiments of this aspect, R_{2′} and R_{3′} are each hydrogen to facilitate di-halogenation of the ring.

In certain embodiments of selectively halogenating, the compound of Formula I′ is dissolved in a solvent prior to contacting the compound of Formula I′ with HX in the presence of DMSO. In certain embodiments, the solvent is selected from the group consisting of ethyl acetate, isopropyl acetate, acetonitrile, acetone, t-butyl methyl ether, ethanol, dichloromethane, n-heptane, toluene, 2-Me-THF, and isopropanol. In certain embodiments, the solvent is selected from the group consisting of ethyl acetate, isopropyl acetate, acetonitrile and acetone. In certain embodiments, the solvent is ethyl acetate.

In certain embodiments of selectively halogenating, the first solvent is ethyl acetate in an amount of at least 15 volumes. However, in certain embodiments, the amount of solvent can be 5 volumes, 6 volumes, 7 volumes, 8 volumes, 9 volumes, 10 volumes, 11 volumes, 12 volumes, 13 volumes, 14 volumes, 15 volumes, 16 volumes, 17 volumes, 18 volumes, 19 volumes, 20 volumes, 21 volumes, 22 volumes, 23 volumes, 24 volumes, 25 volumes, or more. In certain embodiments, the solvent is present in a range from about 5 volumes to about 25 volumes; or about 9 volumes to about 17 volumes; or about 9.7 volumes to about 16.1 volumes. In certain embodiments, the solvent is ethyl acetate in an amount of at least 15 volumes.

When performing the selective halogenation, the amount of organic sulfoxide, such as DMSO, can vary. In certain embodiments, organic sulfoxide, such as DMSO is present in an amount of about 1.5-5.0 molar equivalents. In certain embodiments, organic sulfoxide, such as DMSO is present in an amount of about 1.8-4.5 molar equivalents. In certain embodiments, organic sulfoxide, such as DMSO is present in an amount of about 2.0-3.0 molar equivalents. In certain embodiments, organic sulfoxide, such as DMSO is present in an amount of about 1.9 molar equivalents, 2.0 molar equivalents, 2.1 molar equivalents, or 2.2 molar equivalents.

In certain embodiments of selectively halogenating, the contacting the compound of Formula I′ with a solvent and/or the contacting of the mixture with HX is at a temperature from about 0° C. to about 100° C.; or from about 10° C. to about 90° C.; or from about 20° C. to about 80° C.; or from about 30° C. to about 70° C.; or from about 40° C. to about 60° C.; or from about 45° C. to about 55° C. In certain embodiments of selectively halogenating, the contacting is at a temperature from about 0° C. to about 20° C., from about 20° C. to about 25° C., from about 25° C. to about 30° C., from about 30° C. to about 35° C., from about 35° C. to about 40° C., from about 40° C. to about 45° C., from about 45° C. to about 50° C., from about 50° C. to about 55° C., from about 55° C. to about 60° C., from about 60° C. to about 65° C., from about 65° C. to about 70° C., about 70° C. to about 75° C., from about 75° C. to about 80° C., or from about 80° C. to about 100° C. In certain embodiments, the temperature is from about 40° C. to about 60° C. In certain embodiments, the methods do not include any cooling.

In certain embodiments of selectively halogenating, the contacting the mixture of with HX is for a period of time from about 5 minutes to about 24 hours, or from about 30 minutes to about 20 hours, or from about 30 minutes to about 15 hours, or from about 30 minutes to about 10 hours, or from about 30 minutes to about 5 hours, or from about 30 minutes to about 3.5 hours, or from about 1 hour to about 3 hours, or from about 1.5 hours to about 2.5 hours. In certain embodiments, the contacting is for a period of time of about 5 minutes, about 10 minutes, about 20 minutes, about 30 minutes, about 45 minutes, about 1 hour, about 1.25 hours, about 1.5 hours, about 1.75 hours, about 2 hours, about 2.25 hours, about 2.5 hours, about 2.75 hours, about 3 hours, about 3.25 hours, about 3.5 hours, about 3.75 hours, about 4 hours, about 4.25 hours, about 4.5 hours, about 4.75 hours, about 5 hours, about 10 hours, about 15 hours, about 20 hours, about 24 hours, or more.

In certain embodiments, the method of selectively halogenating produces the compound of Formula I having a purity above about 88A %, above about 90A %, above about 92A %, above about 93A %, above about 94A %, above about 95A %, above about 96A %, above about 97A %, above about 98% or above about 99A %.

In certain embodiments, the method of selectively halogenating produces the compound of Formula I selected from the group consisting of:

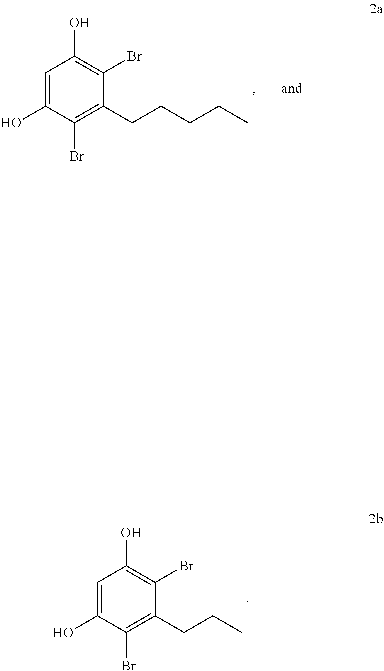

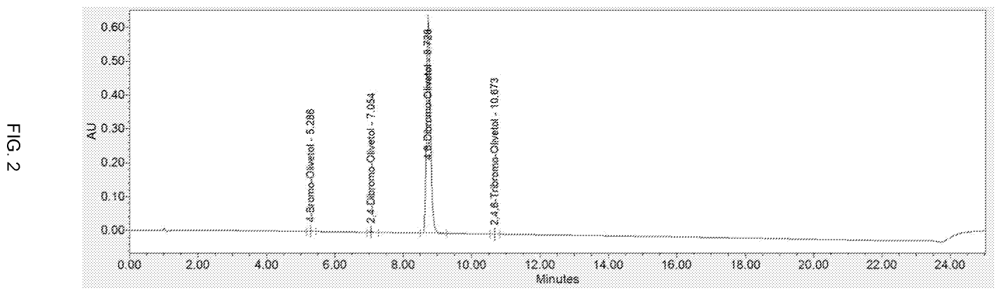

Reaction progress, reaction completion (IPC) and purity can be monitored by HPLC. A representative HPLC of a DBO isolated solid is shown in FIG. 2.

In certain embodiments, quenching of the methods described above comprises, after the contacting step, contacting the reaction mixture comprising a compound of Formula I with a buffered quench solution at a pH of about 14. In certain embodiments, the quench solution comprises K₂HPO₄ and NaOH. In certain embodiments, the quench solution is water, K₂HPO₄ and about 10% to 30% NaOH. In certain embodiments, the buffer solution comprises about 18% NaOH. In certain embodiments, the quench solution is not a NaHCO₃ solution or other solution that results in the problematic evolution of a gas.

In certain embodiments, the methods described above can further comprise a crystallization process.

Unlike the art methods for preparing dibromo-olivetol, which require gaseous diatomic bromine (Br₂) at sub-zero temperatures, Scheme 2 depicts a general procedure synthetic route for preparing the compounds of Formula I using HX in DMSO (or alternative organic sulfoxide) at temperatures above 0° C.

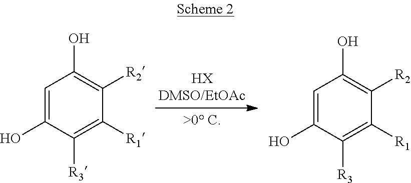

The general reaction scheme includes: charge the dihydroxybenzene, such as olivetol; charge the first solvent; and charge DMSO (or alternative organic sulfoxide) and HX; optionally heating the reaction. The reaction progress was monitored by HPLC.

In certain embodiments, the methods described herein are directed to preparing 4,6-dibromo-olivetol in high yield, with high purity and selectivity. Scheme 3 depicts an exemplary route for such a synthesis.

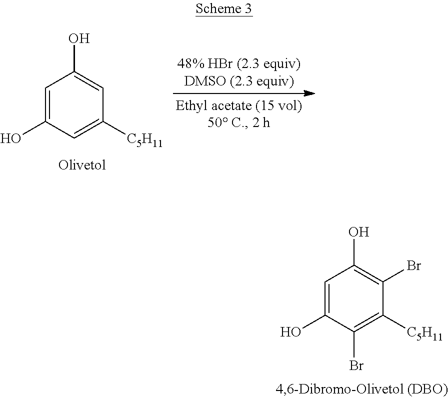

In certain embodiments, the methods described herein are directed to preparing 4,6-dibromo-olivetol in high yield, with high purity and selectivity. Scheme 4 depicts an exemplary route for such a synthesis, further comprising optional purification.

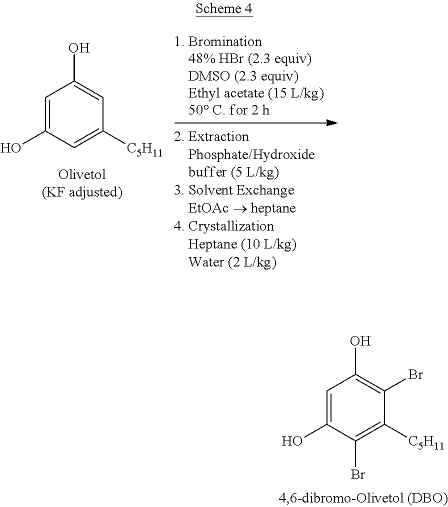

The subject matter described herein includes the following embodiments:
1. A method of preparing a compound of Formula I:

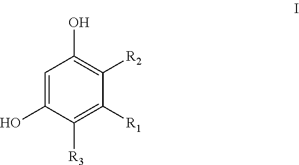

wherein,

R₁ is a branched or straight chain C₁₋₁₂ alkyl; and

R₂ and R₃ are each independently selected from the group consisting of halogen, —C(O)O—C₁₋₆ alkyl, and hydrogen, wherein at least one of R₂ and R₃ is halogen, the method comprising:

contacting a compound of Formula I′ having a structure:

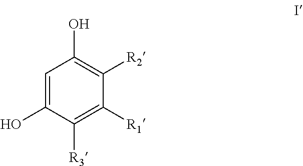

wherein, R_{1′} is a branched or straight chain C₁₋₁₂ alkyl; and
R_{2′} and R_{3′} are each independently selected from the group consisting of halogen, —C(O)O—C₁₋₆ alkyl, and hydrogen, wherein at least one of R_{2′} and R_{3′} is hydrogen;

with HX, wherein X is a halide, in the presence of an organic sulfoxide

wherein, the contacting is at a temperature from about 0° C. to about 100° C.; and

wherein, the compound of Formula I is prepared.
2. The method of embodiment 1, wherein HX is selected from HBr, HCl, HI, and HF.
3. The method of embodiment 1 or 2, wherein HX is HBr.
4. The method of embodiment 1, 2 or 3, wherein the HBr is aqueous.
5. The method of embodiment 1, 2, 3 or 4, wherein R₁ and R_{1′} are the same and each is selected from the group consisting of straight or branched methyl, ethyl, propyl, isopropyl, n-butyl, sec-butyl, isobutyl, t-butyl, pentyl, hexyl, heptyl, octyl, nonyl, decyl, undecyl, and dodecyl.
6. The method of embodiment 1, 2, 3, 4 or 5, wherein R₁ and R_{1′} are each propyl or pentyl.
7. The method of embodiment 1, 2, 3, 4, 5 or 6, wherein R₁ and R_{1′} are the same and each is selected from the group consisting of a branched chain C₁₋₁₂ alkyl having one, two, three, four, five, six, seven, eight, nine, ten, eleven, or twelve carbon atoms.
8. The method of embodiment 1, 2, 3, 4, 5, 6 or 7, wherein the compound of Formula I is a compound having a structure:

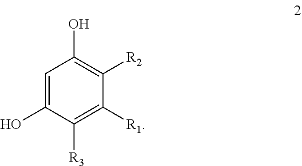

wherein R₁ and R_{1′} are the same.
9. The method of embodiment 1, 2, 3, 4, 5, 6, 7 or 8, wherein R₁ and R_{1′} are each selected from the group consisting of straight or branched methyl, ethyl, propyl, isopropyl, n-butyl, sec-butyl, isobutyl, t-butyl, pentyl, hexyl, heptyl, octyl, nonyl, decyl, undecyl, and dodecyl.
10. The method of embodiment 1, 2, 3, 4, 5, 6, 7, 8 or 9, wherein R₁ and R_{1′} are each selected from the group consisting of propyl and pentyl.
11. The method of embodiment 1, 2, 3, 4, 5, 6, 7, 8, 9 or 10, wherein R₁ and R_{1′} are each pentyl.
12. The method of embodiment 1, 2, 3, 4, 5, 6, 7, 8, 9, 10 or 11, wherein the compound of Formula I is a compound having a structure:

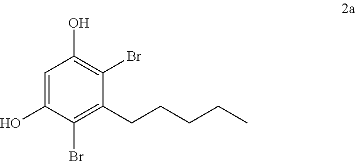

wherein said compound has a purity above about 93A % by HPLC.
13. The method of embodiment 1, 2, 3, 4, 5, 6, 7, 8, 9, 10, 11 or 12, wherein the purity is about 93A % to about 99.5A % by HPLC.
14. The method of embodiment 1, 2, 3, 4, 5, 6, 7, 8, 9, 10, 11, 12 or 13, wherein the purity is about 94A % to about 99.5A %.
15. The method of embodiment 1, 2, 3, 4, 5, 6, 7, 8, 9, 10, 11, 12, 13 or 14, wherein the compound of Formula I′ is selectively di-halogenated in the 4 and 6 positions.
16. The method of embodiment 1, 2, 3, 4, 5, 6, 7, 8, 9, 10, 11, 12, 13, 14 or 15, wherein the 4,6-di-halogenated compound of Formula I, wherein each of R₁ and R₂ is halogen is prepared at a ratio of from about 25:1 to about 34:1 relative to the 2,4-dihalogenated impurity compound.
17. The method of embodiment 1, 2, 3, 4, 5, 6, 7, 8, 9, 10, 11, 12, 13, 14, 15 or 16, wherein prior to said contacting, the compound of Formula I′ is contacted with a first solvent to form a mixture.
18. The method of embodiment 1, 2, 3, 4, 5, 6, 7, 8, 9, 10, 11, 12, 13, 14, 15, 16 or 17, wherein the first solvent is selected from the group consisting of ethyl acetate, isopropyl acetate, acetonitrile, acetone, t-butyl methyl ether, ethanol, dichloromethane, n-heptane, toluene, 2-Me-THF, and isopropanol.
19. The method of embodiment 1, 2, 3, 4, 5, 6, 7, 8, 9, 10, 11, 12, 13, 14, 15, 16, 17 or 18, wherein the solvent is selected from the group consisting of ethyl acetate, isopropyl acetate, acetonitrile, and acetone.
20. The method of embodiment 1, 2, 3, 4, 5, 6, 7, 8, 9, 10, 11, 12, 13, 14, 15, 16, 17, 18 or 19, wherein the solvent is present from about 9.0 vol to about 17 vol.
21. The method of embodiment 1, 2, 3, 4, 5, 6, 7, 8, 9, 10, 11, 12, 13, 14, 15, 16, 17, 18, 19 or 20, wherein the solvent is present from about 9.7 vol to about 16.1 vol.
22. The method of embodiment 1, 2, 3, 4, 5, 6, 7, 8, 9, 10, 11, 12, 13, 14, 15, 16, 17, 18, 19, 20 or 21, wherein the organic sulfoxide is present in an amount of about 2.0 equiv to about 3.0 equiv.
23. The method of embodiment 1, 2, 3, 4, 5, 6, 7, 8, 9, 10, 11, 12, 13, 14, 15, 16, 17, 18, 19, 20, 21 or 22, wherein the HX is present in an amount from about 2.0 equiv to about 3.0 equiv.
24. The method of embodiment 1, 2, 3, 4, 5, 6, 7, 8, 9, 10, 11, 12, 13, 14, 15, 16, 17, 18, 19, 20, 21, 22 or 23, wherein the yield is above about 82%.
25. The method of embodiment 1, 2, 3, 4, 5, 6, 7, 8, 9, 10, 11, 12, 13, 14, 15, 16, 17, 18, 19, 20, 21, 22, 23 or 24, wherein the yield is from about 82% to about 90%.
26. The method of embodiment 1, 2, 3, 4, 5, 6, 7, 8, 9, 10, 11, 12, 13, 14, 15, 16, 17, 18, 19, 20, 21, 22, 23, 24 or 25, wherein the yield is above about 95%.
27. A method of preparing a compound of Formula I:

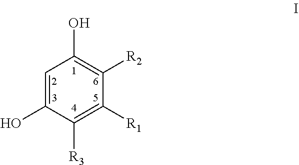

wherein,

R₁ is a branched or straight chain C₁₋₁₂ alkyl; and

R₂ and R₃ are each halogen,

the method comprising:

selectively halogenating at the 4- and 6-positions by contacting a compound of Formula I′ having a structure:

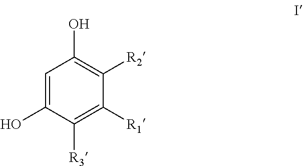

wherein, R_{1′} is a branched or straight chain C₁₋₁₂ alkyl; and

R_{2′} and R_{3′} are each hydrogen;

with a first solvent to form a mixture,

contacting the mixture with HX, wherein X is a halide, in the presence of an organic sulfoxide;

wherein, the contacting is at a temperature from about 0° C. to about 100° C.; and

wherein, the compound of Formula I is prepared.
28. The method of embodiment 27, wherein the compound of Formula I is present at a ratio of at least 10:1 relative to a mono-halogenated, tri-halogenated or 2,4-dihalogenated compound.
29. The method of embodiment 27 or 28, wherein the ratio is at least 20:1.
30. The method of embodiment 27, 28 or 29, wherein the ratio is from about 25:1 to about 35:1.
31. The method of embodiment 27, 28, 29 or 30, wherein the halide is Br and HX is HBr.
32. The method of embodiment 27, 28, 29, 30 or 31, wherein the HBr is an aqueous solution.
33. The method of embodiment 27, 28, 29, 30, 31 or 32, wherein the first solvent is ethyl acetate in an amount of at least 15 volumes.
34. The method of embodiment 27, 28, 29, 30, 31, 32 or 33, wherein the contacting is at a temperature from about 35° C. to about 60° C.
35. The method of embodiment 27, 28, 29, 30, 31, 32, 33 or 34, wherein the compound of Formula I has a purity above about 99A %.
36. The method of embodiment 27, 28, 29, 30, 31, 32, 33, 34 or 35, wherein R_{1′} is propyl or pentyl, and the compound of Formula I is selected from the group consisting of:

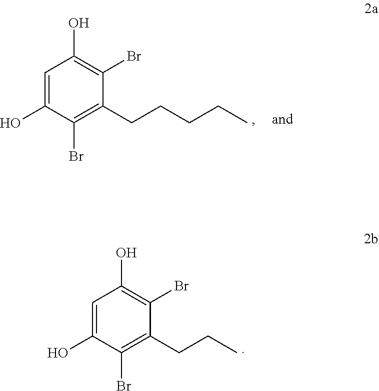

37. The method of embodiment 1 or 27, further comprising:

after said contacting for said period of time, aqueous NaOH is added to the reaction.
38. The method of embodiment 37, wherein said aqueous NaOH is a 10% to 50% (v/v) solution.
39. The method of embodiment 38, wherein said aqueous NaOH is about 18% (v/v) solution
40. A composition comprising 4,6-DBO; 4-MBO; 2,4-DBO and TBO in amounts of about 94%, about 3%, about 1%, and about 1%, respectively.
41. The method of embodiment 1 or 27, wherein said organic sulfoxide is of the general formula:

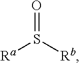

wherein, Ra and Rb are each independently selected from the group consisting of benzyl, phenyl, alkyl, aryl and allyl.
42. The method of embodiment 41, wherein at least one of Ra and Rb is C₁₋₆ alkyl.
43. The method of embodiment 42, wherein the organic sulfoxide is DMSO.

The present invention is further described in the following non-limiting Examples. It should be understood that these Examples, while indicating preferred embodiments of the invention, are given by way of illustration only.

### EXAMPLES

### Example 1: Method A

This process involved dissolving olivetol (not corrected for water %) in ethyl acetate (22 mL/g) and then adding aqueous 48% HBr (2.1 equiv.) and DMSO (2.1 equiv.) at 60° C. for 1 h. This procedure yielded the impurity 2,4,6-tribromo-olivetol (TBO) at high levels in the reaction (18.7%) and a modest yield of DBO (65%).

Subsequently, several reaction parameters were systematically tested. These include solvent volume, HX equivalents, reaction temperature, and review of impurities profile. Advantageously, it was found that certain parameters can be tuned to provide the desired compounds of Formula I at high purity and yields.

### Example 2: Method B

This process involved reducing the level of tri-halogenated compounds. The reaction temperature was decreased to about 30° C. (Method B, Table 1). The results indicated the reaction completed at 30-40° C. in 6 h. The TBO impurity was found to be 0.7A % at the IPC. The reaction was isolated according to the saturated NaHCO₃ quench method (see Example 6) in 87% yield and 99.8A %. The TBO level was reduced to 0.1A %. These modified conditions provided a marked improvement. The results are summarized in Table 1.

TABLE 1

| | DMSO and | | | In-Process Control (IPC) HPLC results (A %) | | | | |
|---|---|---|---|---|---|---|---|---|
| | 48% HBr | Reaction | Time | 4- | 2,4- | 4,6- | | # unknown |
| Method | (equiv.) | Temp | (h) | MBO | DBO | DBO | TBO | Peaks (≥0.5) |
| A | 2.1 | 60° C. | 1.0 | 0.0 | 0.0 | 79.6 | 18.7 | 3 |
| | | isolated | NA | 0.0 | 0.0 | 72.7 | 27.3 | 1 |
| | | DBO | | | | | | |
| | | (yield 65%) | | | | | | |
| B | 2.1 | 30° C. | 3.5 | 7.2 | 2.8 | 89.7 | 0 | 1 |
| | | increased to | +2.5 | 0.1 | 3.0 | 96.1 | 0.8 | 1 |
| | | 40° C. | | | | | | |
| | | isolated | NA | 0.0 | 0.1 | 99.8 | 0.1 | 1 |
| | | DBO (yield | | | | | | |
| | | 87%) | | | | | | |

### Example 3: Testing of Solvent Volumes

This process involved a decrease in reaction volume. To study the effect of a decrease in reaction volume, the experiments were conducted using 15 volumes of solvent (ethyl acetate). The results from experiment 5 (Table 2) indicated that the reaction was sluggish to go to completion at 35° C. with 2.1 equivalents of HBr even after 7 hours as there was 3.5% 4-MBO remaining. Increasing the reaction temperature to 50° C. caused the reaction to go to completion when using 2.2 or 2.3 equivalents of HBr. Since 2.3 equivalents of HBr produced a lower amount of impurity 4-MBO (0.6 A %), these conditions were selected for additional modifications. Experiment 13 conditions were used for evaluating the work up procedure and subsequent scale up. The results are shown in Table 2.

TABLE 2
Ethyl Acetate 15 g/mL, IPC Data

| | DMSO and | | | IPC HPLC results (A %) | | | | |
|---|---|---|---|---|---|---|---|---|
| Experiment | 48% HBr | Reaction | Time | 4- | 2,4- | 4, 6- | | # unknown |
| Number | (equiv.) | Temp | (h) | MBO | DBO | DBO | TBO | Peaks (≥0.5) |
| 5 | 2.1 | 35° C. | 1 | 32.4 | 2.1 | 65.0 | ND^{a} | 2 |
| | | | 7 | 3.5 | 2.8 | 93.0 | 0.2 | 5 |
| 10 | 2.2 | 50° C. | 1 | 2.1 | 3.6 | 94.0 | 0.3 | 0 |
| | | | 2/3 | 0.8 | 3.6 | 94.0 | 1.4 | 0 |
| 13 | 2.3 | 50° C. | 1 | 0.8 | 3.7 | 95.0 | 0.6 | 0 |
| | | | 2 | 0.6 | 3.7 | 94.0 | 1.6 | 0 |

Further reduction of solvent volume was tested. The amount of ethyl acetate was reduced to 10 volumes (Table 3, Experiment 6). Using the optimized reaction conditions for 22 volumes of ethyl acetate (2.3 equiv. HBr, 50° C.), the reaction was found to not progress as effectively when using 10 volumes solvent. After 7 hours, the reaction showed an 89.0A % of DBO and a 7.0A % of 4-MBO. Increasing the temperature, reaction time and equivalents of HBr (Table 3, Experiment 9 and Experiment 17) did recover some of the yield of DBO, but the amount of 4-MBO was above 1.0%. The data indicate that the reaction is slower when using 10 volumes of ethyl acetate. Based on the testing, 15 volumes solvent was selected as the lowest concentration that the reaction conditions could tolerate without sacrificing yield and purity of DBO. The results are shown in Table 3.

TABLE 3
Ethyl Acetate 10 g/mL, IPC Data

| | DMSO and | | | IPC HPLC results (A %) | | | | |
|---|---|---|---|---|---|---|---|---|
| Experiment | 48% HBr | Reaction | Time | 4- | 2,4- | 4,6- | | # unknown |
| Number | (equiv.) | Temp | (h) | MBO | DBO | DBO | TBO | Peaks (≥0.5) |
| 6 | 2.1 | 40° C. | 1 | 25.0 | 2.5 | 72.0 | ND^{a} | 1 |
| | | | 7 | 7.0 | 3.4 | 89.0 | 0.2 | 1 |
| 9 | 2.1 | 60° C. | 1 | 5.0 | 4.4 | 90.0 | 0.3 | 1 |
| | | | 2 | 4.3 | 4.8 | 90.3 | 0.6 | 1 |
| | +0.2 | | +1 | 1.1 | 5.1 | 91.0 | 2.8 | 2 |
| 17 | 2.35 | 50° C. | 1 | 3.7 | 3.8 | 92.0 | 0.2 | 1 |
| | | | 2 or 3 | 2.2 | 4.2 | 93.0 | 0.8 | 1 |
| | +0.11 | | +1 | 1.3 | 4.5 | 92.0 | 1.7 | 2 |

### Example 4: Solvent Screening

After several process variables, such as solvent volume, temperature, time, and equivalents of HX (e.g., HBr) had been tested, several solvents were screened to assay which solvents performed as well or better than ethyl acetate. The solvent screening was conducted according to the following procedure.

Olivetol (1 g, corrected for water%) and solvent (15 vol.) were charged to a 40 mL vial. To the solution was added DMSO (2.3 equiv.) and dropwise 48% HBr (2.3 equiv.). The reaction was heated to 50° C. and aliquots were taken for analysis at 2, 6, and 22 hours. The reaction progress was monitored by HPLC. The results of the solvent screen are summarized in Table 4.

TABLE 4
Solvent Screening for DBO Reaction

| | DBO Formation (A %) | | |
|---|---|---|---|
| Solvents | 2 h | 6 h | 22 h |
| Ethyl Acetate | 94.8 | 90.5 | 79.2 |
| Isopropyl Acetate | 77.9 | 94.0 | 79.0 |
| Acetonitrile | 89.6 | 89.1 | 84.1 |
| Acetone | 90.3 | 91.1 | 87.9 |
| t-Butyl Methyl Ether | 30.3 | 36.0 | 64.4 |
| Ethanol | 9.4 | 31.0 | 53.7 |
| Dichloromethane | 10.3 | 18.1 | 30.9 |
| n-Heptane | 72.5 | 65.8 | 62.08 |
| | | (solid precipitated) | (solid precipitated) |
| Toluene | 28.6 | 41.5 | 54.2 |
| 2-Me-THF | 63.8 | 78.2 | 85.8 |
| Isopropanol | 9.0 | 38.7 | 58.8 |

The data indicate that four solvents, ethyl acetate, isopropyl acetate, acetonitrile and acetone, each provided ≥89 A % of DBO after 6 hours. The detailed impurity results are shown in Table 5.

TABLE 5
Solvents Ethyl Acetate, Isopropyl Acetate, Acetonitrile and Acetone Impurity Profile

| | | In-Process Control (IPC) HPLC results (A %) | | | | |
|---|---|---|---|---|---|---|
| | Time | | | | | # unknown |
| Solvents | (h) | 4-MBO | 2,4-DBO | 4,6-DBO | TBO | Peaks (≥0.5) |
| Ethyl | 2 | 0.6 | 3.2 | 94.8 | 1.4 | 1 |
| Acetate | 6 | 0.6 | 3.2 | 90.5 | 5.75 | 1 |
| | 22 | 0.9 | 4.1 | 79.2 | 15.7 | 2 |
| Isopropyl | 2 | 18.0 | 3.6 | 77.9 | 0.2 | 2 |
| Acetate | 6 | 0.9 | 4.1 | 94.0 | 0.9 | 1 |
| | 22 | 0.6 | 3.3 | 79.0 | 17.3 | 0 |
| Acetonitrile | 2 | 7.2 | 2.6 | 89.9 | 0.2 | 1 |
| | 6 | 6.9 | 3.5 | 89.1 | 0.4 | 0 |
| | 22 | 6.5 | 7.4 | 84.1 | 1.7 | 1 |
| Acetone | 2 | 9.4 | 2.1 | 87.9 | 0.3 | 1 |
| | 6 | 7.1 | 1.6 | 91.1 | 0.1 | 1 |
| | 22 | 8.1 | 1.4 | 90.1 | 0.1 | 1 |

Overall, a preferred impurity profile was obtained using ethyl acetate (4-MBO NMT 1.0A %). The testing also indicated isopropyl acetate produced a good impurity profile.

### Example 5: Work Up and Isolation

Olivetol (10.0 g), ethyl acetate (220 mL), 48% HBr (19.7 g), and DMSO (9.1 g) were charged to a 500 mL reactor and heated to 60° C. The reaction was sampled for completion after one hour at 60° C. (0.4% MBO; 4.1% 2,4-DBO; 0.8% TBO). The solution was then distilled to 5 pot volumes. To this solution, n-Heptane (300 mL) and water (20 mL) were charged to the reactor. The solution was biphasic with no solid. The mixture was distilled to 20 pot volumes. During the distillation a slurry of crystalline solids were formed. The mixture was cooled to 20° C. and held for 100 minutes before the solids were isolated by filtration over filter paper. The solids were washed with room temperature n-heptane (30 g) dried under vacuum at 40° C. overnight. The isolated yield was 15.4 g (82%); KF 1988.7 ppm; total purity 99.2% by HPLC.

The above procedure was repeated as described above in Method A. After workup, the purity was 72.6% with 27.2 A % TBO formation in 65% yield. See Table 1, Method A.

### Example 6: Reaction Quench Studies

To further improve the methods, the work up and isolation procedure was modified in the workup of Experiment 17 as described below.

Experiment 17 was worked up according to the following modified buffer quench procedure. The buffer solution was prepared in another vessel by combining water (4.55 S (S means scale factor)), K₂HPO₄ (1.45 S) and 30% NaOH (1.11 S) and mixing until dissolved. The pH of the buffer solution was found to be 14.

Once complete, the reaction solution was cooled to room temperature and buffer solution was transferred into the reaction mixture. The solution was stirred for 30 minutes, and then the phases were separated. The aqueous layer (pH 5-6) was discarded. The organic phase was distilled to 5 volumes under vacuum at 45° C. Heptane (2×10 vol) was charged to the reactor, and the solution was distilled to 10 volumes under reduced pressure after each addition.

The distillation set up was replaced and replaced with a condenser. The reaction solution was heated to 50° C. and water (2 vol) was added dropwise. Agitation was continued at 50° C. for another hour after which the reaction was cooled to 20° C. and agitated for an additional 2 h. The slurry was filtered and rinsed with heptane (2.5 vol). The wet cake was dried at 40° C. overnight. The product yield was 16.2 g (92%) (KF=0.2%; purity=99.1 A %, major impurity 4-MBO 0.62A %, ROI 0.7%). The reaction was conducted using 10 g of olivetol (KF 6.3%). The reaction Vₘᵢₙ is 5 volumes and the Vₘₐₓ is 23 volumes.

Experiment 10 used NaHCO₃ as a quench solution. Experiment 10 was worked up according to the procedure below. After completion of the reaction, the reaction mixture was cooled to room temperature. Saturated NaHCO₃ solution (5 g/mL) was added to the reaction mixture, agitated for 30 min and then the phases were separated (mild off gassing of CO₂ gas was observed). The aqueous phase was discarded (pH ˜5). The organic phase was washed with water and distilled to 5 volumes under vacuum, and then heptane (2×15 vol) was charged to the reactor and distilled to 15 volumes after each addition.

The distillation set up was removed and replaced with a condenser. The reaction solution was heated to 50° C. and water (2 vol) was added dropwise. Agitation was continued at 50° C. for another hour after which the reaction was cooled to 20° C. and agitated for an additional 2 h. The slurry was filtered and rinsed with heptane (2 vol). The wet cake was dried at 40° C. overnight. The product yield was 7.3 g (87%) of DBO (KF=0.1%; purity=99.1 A %, major impurity 4-MBO=0.7A %). The reaction was conducted using 5 g of olivetol (KF=10.5%). The reaction Vₘᵢₙ is 5 volumes and the Vₘₐₓ is 20 volumes.

While both methods gave excellent results, because the NaHCO₃ quench reaction resulted in CO₂ gas evolution and it is desired to avoid this method for large scale manufacturing, the modified buffer quench was selected for scale up.

### Example 7: Scale Up Studies

The process was scaled up to 10 g and 2×40 g batches. The results indicated that the process was reproducible and obtained excellent purity of DBO and yield range 87-92% (Table 6). The solvent swap distillation volume and wet cake wash volume was found to affect yield.

Experiment 20 was conducted in a 250 mL non-gradated jacketed flask and it was therefore difficult to measure the solvent volume during the solvent swap (1^{st} distillation 5 vol, 2^{nd} distillation 10 vol. and 3^{rd} distillation 10 vol.). It is noted that some measurement error may have occurred.

Experiment 21 was conducted in a 1 L gradated cylindrical jacked reactor and it was easy to measure solvent volume during the solvent swap (1^{st} distillation 5 vol, 2^{nd} distillation 10 vol. and 3^{rd} distillation 10 vol). The isolated yield of DBO was 87%.

Experiment 22 was conducted in a gradated 1 L cylindrical jacked reactor (1st distillation 5 vol, 2nd distillation 5 vol. and 3rd distillation 10 vol). The isolated DBO yield was 90%. The only difference between Experiments 21 and 22 is the 2nd solvent swap volume. Experiment 21 used 10 volume distillation, while Experiment 22 used 5 vol distillation. It is possible the decreased yield for Experiment 21 is due to the solvent swap not being fully completed. Since DBO is highly soluble in ethyl acetate, Experiment 21 produced a lower yield. Based on the data of Experiment 22, the 5 volumes for the 2^{nd} distillation is recommended for future scale-up. The data are shown in Table 6.

TABLE 6
DBO Scale Up Results

| | Olivetol | | HPLC | 4-MBO | Water |
|---|---|---|---|---|---|
| Experiment | quantity | | Purity | impurity | % |
| Number | (g) | Yield | (A %) | (A %) | (KF) |
| 20 | 10 | 92 | 99.23 | 0.44 | 0.06 |
| 21 | 40 | 87 | 99.79 | 0.12 | 1.03 |
| 22 | 40 | 90 | 99.73 | 0.21 | 0.49 |

Experiment 23 was a scale-up to 40 g starting material that was conducted to overcome salt formation observed during the quench procedure.

In experiment 23-A, Olivetol (40 g, not corrected for water%) and solvent (15 vol.) were charged to a 1 Liter reactor. To the solution was added DMSO (2.3 equiv.) and 48% HBr (2.3 equiv.) was charged in a controlled manner. The reaction was heated to 50° C. under agitation and aliquots were taken for HPLC analysis at 0.5, 1, 1.5, and 2 hours. The HPLC results are shown in Table 7.

TABLE 7
HPLC data for Experiments 23-A and 23-B

| | Ethyl | | Reaction | HPLC result (A %) | | | | | | |
|---|---|---|---|---|---|---|---|---|---|---|
| Exp. | acetate | HBr and | Temp | | | 2- | 4- | 2,4- | 4,6- | |
| number | (L/kg) | DMSO Eq. | (° C.) | Time (min) | Olivetol | MBO | MBO | DBO | DBO | TBO |
| 23-A | 15 | 2.30 | 50 | 0 | 0 | 0.66 | 29.3 | 3.4 | 66.6 | 0.06 |
| | | | | 30 | 0 | 0.05 | 2.22 | 4.5 | 92.7 | 0.5 |
| | | | | 60 | 0 | 0 | 0.41 | 4.4 | 93.4 | 1.8 |
| | | | | 90 | 0 | 0 | 0.28 | 4.2 | 92.5 | 3.1 |
| | | | | 120 | 0 | 0 | 0.25 | 3.8 | 89.8 | 6.2 |
| 23-B | 15 | 2.30 | 50 | 0 | 0.04 | 0.9 | 47.9 | 2.3 | 48.8 | 0 |
| | | | | 30 | 0 | 0 | 0.05 | 3.4 | 3.9 | 92.6 |
| | | | | 60 | 0 | 0 | 0 | 1.2 | 4.3 | 94.2 |
| | | | | 90 | 0 | 0 | 0.8 | 4.2 | 94.3 | 0.66 |
| | | | | 120 | 0 | 0 | 0.7 | 4.1 | 94.1 | 1.16 |

At the end of 2 hours, the reaction mixture was cooled to 20° C. HPLC samples were collected. A buffer solution consisting of 30% NaOH, potassium phosphate dibasic, and de-ionized water in the amount of 1.11 eq., 1.45 eq., and 4.55 eq. respectively was used to quench the bromination reaction. The pH of the buffer solution was measured to be 12.74. The buffer solution was added to bring the reaction mixture to a final pH of 5.68 as recommended earlier. The reaction mixture was distilled under vacuum to 5 L/kg. The solution appeared bi-phasic. To the biphasic solution was then added 10 L/kg n-heptane and the reaction mixture was distilled under vacuum again to 5 L/kg. Another charge of heptane was added to the reaction mixture and distilled under vacuum to 10 L/kg. A slurry of solids was observed. The solution was cooled to 50° C. and water was added. The solids in the solution crystallized instantaneously and the mixture was aged (under agitation) for 1 hour. The solution was then cooled to 20° C. and filtered. Solids were isolated over filter paper. The solid cake was washed with 3 L/kg heptane and isolated solids were dried under vacuum at 45° C. overnight.

The addition of the buffer solution led to a 4° C. rise in the temperature of the reaction mixture. Upon cooling the reaction mixture to 20° C., a noticeable amount of salt formation in the aqueous phase of the (quenched) reaction mixture. To address the salt formation issue, DI water was selected to add to the reaction mixture with agitation until the salt appeared dissolved. The remaining unit operations consisting of solvent-swap distillation, crystallization, and filtration was carried out based on the usual procedure.

In Experiment 23-B, the reaction was performed under identical conditions as described above for Experiment 23-A and sampled for LC as shown in Table 8. In experiment 23-B, however, a buffer solution consisting of 30% NaOH was used. The final amount of 30% NaOH used was 2.75 Eq. to achieve a pH of 5.68 as compared to the experiment 23-A, which used 1.11 Eq. No salt formation was observed when 30% NaOH was used.

TABLE 8
Yield and HPLC data for isolated product for Experiments A and B

| Exp. | Dry DBO | HPLC result for isolated product (A %) | | | | | |
|---|---|---|---|---|---|---|---|
| number | yield solids | Olivetol | 2-MBO | 4-MBO | 2,4-DBO | 4,6-DBO | TBO |
| 23-A | 90.5% | 0 | 0 | 0.33 | 0.04 | 99.4 | 0.11 |
| 23-B | 88.3% | 0 | 0 | 0.11 | 0.03 | 99.08 | 0.77 |

Experiments 23-A and 23-B gave product of acceptable quality and yield (Table 8) and show that the adjusted parameters overcame the salt formation in Experiment 23-A. Thus, the processes can be completed without substantial salt formation.

Experiment 24 was run to determine DBO product and impurity evolution profiles: Effect of temperature hold at various stages of HBr addition. Determining DBO product and impurity evolution profiles (i) during HBr addition programmed to occur over 30 minutes; (ii) the effect of holding temperature at 20° C. for 2 h after HBr is added; (iii) after holding at 20° C. for 2 h, reaction mixture was heated to 50° C. In this experiment, Olivetol (6 g, not corrected for water%) and solvent (15 vol.) were charged to a 100 mL reactor. To the solution was added DMSO (2.3 equiv.) and 48% HBr (2.3 equiv.) was charged in a controlled manner. The reaction was heated to 50° C. under agitation and aliquots were taken for HPLC analysis at 0.5, and 1 hour during HBr addition at 20° C.; sampled at 0.5, 1, 1.5, and 2 hours at 20° C. after HBr addition; sampled at 0.5, 1, 1.5, 2, 2.5, 5, and 24 hours at 50° C. after HBr addition. HPLC results are shown in Table 9.

TABLE 9
HPLC data for temperature hold at various stages of HBr reagent addition

| | | Temp | A % | A % (2- | A % (4- | A % (2,4- | A % (4,6- | A % (2,4,6- |
|---|---|---|---|---|---|---|---|---|
| Step | Time | ° C. | (Olivetol) | MBO) | MBO) | DBO) | DBO) | TBO) |
| 20° C. DURING | −10+ | 20 | 100 | 0 | 0 | 0 | 0 | 0 |
| HBr addition | 0 | 20 | 80.64 | 0.38 | 18.91 | 0.01 | 0.05 | 0.02 |
| | 30 | 20 | 55.72 | 1.29 | 42.73 | 0.03 | 0.24 | 0 |
| | 60 | 20 | 27.39 | 1.85 | 69.94 | 0.06 | 0.77 | 0 |
| 20° C. | 30 | 20 | 0.11 | 2.24 | 92.07 | 0.25 | 5.33 | 0 |
| (AFTER HBr) | 60 | 20 | 0.06 | 2.02 | 79.38 | 0.69 | 17.84 | 0 |
| | 90 | 20 | 0.05 | 1.7 | 67.63 | 1.07 | 29.55 | 0 |
| | 120 | 20 | 0.04 | 1.42 | 59.96 | 1.3 | 37.28 | 0 |
| 50° C. | 0 | 50 | 0.02 | 0.58 | 27.57 | 2.47 | 69.29 | 0.04 |
| (AFTER 20° C.) | 30 | 50 | 0 | 0.02 | 1.7 | 3.48 | 94.53 | 0.2 |
| | 60 | 50 | 0 | 0.01 | 0.74 | 3.49 | 95.24 | 0.52 |
| | 90 | 50 | 0 | 0 | 0.63 | 3.53 | 94.89 | 0.94 |
| | 120 | 50 | 0 | 0 | 0.57 | 3.48 | 94.51 | 1.44 |
| | 150 | 50 | 0 | 0 | 0.55 | 3.45 | 94.05 | 1.95 |
| | 900 | 50 | 0 | 0.01 | 0.81 | 3.7 | 82.66 | 12.83 |
| | 1515 | 50 | 0 | 0.01 | 0.95 | 4.16 | 78.49 | 16 |

^{†}Time at which no HBr has been added to the reactor relative to the starting time for HBr addition (0 min, in this case)

Results indicate that the bromination reaction starts almost instantaneously after adding the HBr. The kinetics of the bromination of olivetol are slow at 20° C. during HBr addition. However, once complete addition of HBr is achieved, the olivetol is consumed and the reaction was found to be highly selective for 4,6-Dibromo Olivetol (4,6-DBO) formation. Maximum 4,6-DBO was formed at the end of 1.5 hours at the reaction temperature of 50° C. The reaction was allowed to proceed at 50° C. for 24 hours. This extended period led increased levels of 2,4-DBO and TBO levels with a steady decrease of 4,6-DBO. The level of 4-MBO reached a maximum at 1.5 hours and then steadily decreased with time.

### Example 8: General Synthetic Procedure for Preparing 4,6-Dibromo-Olivetol

1. Process description for Preparing DBO: All charges are based off of corrected weight of olivetol.
Set up 1 L jacketed reactor with mechanical stirrer, condenser, thermocouple and a nitrogen atmosphere. Set jacket temperature to 20-25° C.
Charge Olivetol (40 g, 1 equiv., KF 6.3%, corrected weight 37.48 g).
Charge Ethyl Acetate (562 mL, 15 vol.) and agitate at room temperature.
Charge Dimethyl sulfoxide (DMSO, 37.4 g, 2.3 equiv.).
Charge aqueous 48% Hydrobromic Acid¹ (80.6 g, 2.3 equiv.) dropwise over 10 minutes.
Heat to 50-52° C. for 2 h².
IPC for remaining 4-MBO ≤1%.

2. Work Up
Cool the reaction to 25° C.
In a second appropriately sized reaction vessel, prepare buffer solution³ containing water (43 mL, 4.55 S⁴), potassium phosphate dibasic (13.6 g, 1.45 S) and 30% NaOH (6.3 g, 1.11 S). This solution is exothermic (60° C.) and prepared in advance (1 h) and cool to ˜25° C.
Add buffer solution to the reaction mixture and agitate for 30 min. No exotherm was observed upon addition of the buffer solution.
Remove bottom aqueous phase⁵.
Set up distillation and distill to 5 vol. (200 mL) under vacuum⁶.
Add heptane (375 mL, 10 vol.) and distill to 5 vol⁷.
Add heptane (375 ml, 10 vol.) and distill to 10 vol⁸.
Remove distillation condenser and replace with water condenser.
Heat solution to 50° C. and add water (75 mL, 2 vol.) dropwise and agitate for 1 h⁹.
Cool to 20° C. and agitate for 2 h.
Filter¹⁰ the slurry and rinse with heptane 3 vol. (110 mL).
Dry wet cake at 40° C. for no less than 12 hours under vacuum at 0 to 200 mbar until the LOD ≤0.5%.
DBO was obtained as a white, crystalline solid in 90% (63.5 g) Yield and HPLC purity 99.73 A %.
The reaction has a Vₘᵢₙ=5 and Vₘₐₓ=23.
   It should be noted that:
1. HBr addition is slightly exothermic (max 4° C.).
2. Typical reaction time is 1 h, IPC sample preparation: Take 6 μl of aliquot dissolved in 1 mL of acetonitrile-water (7:3) mixture.
3. Buffer solution preparation is exothermic (˜50° C.), prepare solution at least 1 h before use.
4. Olivetol (corrected for water %) is used as the scale factor.
5. Clear and quick phase separation was observed, make sure all aqueous phase removed (otherwise higher ROI in the isolated DBO), aqueous phase pH 5-6.
6. Set up jacket temperature to 50° C. and vacuum 20-25 in Hg, ethyl acetate and distill at ˜26° C.
7. Solvent distills at 26-29° C., since DBO is highly soluble in ethyl acetate, complete solvent swap is important.
8. Solvent distills at 38-41° C.
9. Easily stirrable slurry.
10. Filtration is rapid.

### Example 9: Stress Reaction

The optimized reaction conditions described in Examples 7 and 8 were used to examine the effect of reaction time on the impurity profile. The HPLC results are summarized in Table 10. The reaction completed in an hour (4-MBO NMT 1.0 A %) and continued the reaction another 21 hours to observe impurity profile. After 22 h the reaction generated 15.7 A % TBO impurity. The TBO impurity will purge during the isolation. These results indicated that the reaction is robust and if the reaction time is extended, high quality material can still be isolated. The data are shown in Table 10.

TABLE 10
Stress Reaction for DBO and IPC Data

| | DMSO | | | | | | | |
|---|---|---|---|---|---|---|---|---|
| | and | | | IPC HPLC results (A %) | | | | |
| Experiment | HBr | Reaction | Time | | 2,4- | 4, 6- | | # unknown |
| Number | (equiv.) | Temp | (h) | 4-MBO | DBO | DBO | TBO | Peaks (≥0.5) |
| 19 | 2.3 | 50° C. | 1 | 0.9 | 3.2 | 95.2 | 0.6 | 0 |
| | | | 2 | 0.6 | 3.2 | 94.8 | 1.4 | 1 |
| | | | 6 | 0.6 | 3.2 | 90.5 | 5.6 | 1 |
| | | | 22 | 0.9 | 4.1 | 79.2 | 15.7 | 0 |

### Example 10: Further Assessment of Modified Reaction Conditions

A further set of experiments were conducted to analyze the amount of ethyl acetate, amount of reactive species, HBr addition rate, excess reagent, excess amount, and reaction temperature. The following general sampling/temperature profile was used
Held reactor temperature at 20° C. during the HBr addition
Sample collection #1
Aged for 30 min at 20° C. after the HBr addition
Sample collection #2
Heated to reaction temperature over 15 min
Sample collection #3
Sample #4 after 30 min, Sample #5 after 60 min, Sample #6 after 120 min, Sample #7 after 240 min, Sample #8 after 480 min

### Constants

1. Agitation Rate (700 rpm)

2. Reaction Time (6 h)

3. Temperature during HBr addition (20° C.)

4. Hold time after HBr addition (30 min)

### Responses

1. 4,6-DBO at 6 h

2. 2,4-DBO at 6 h

3. TBO at 6 h

4. Maximum 4,6-DBO

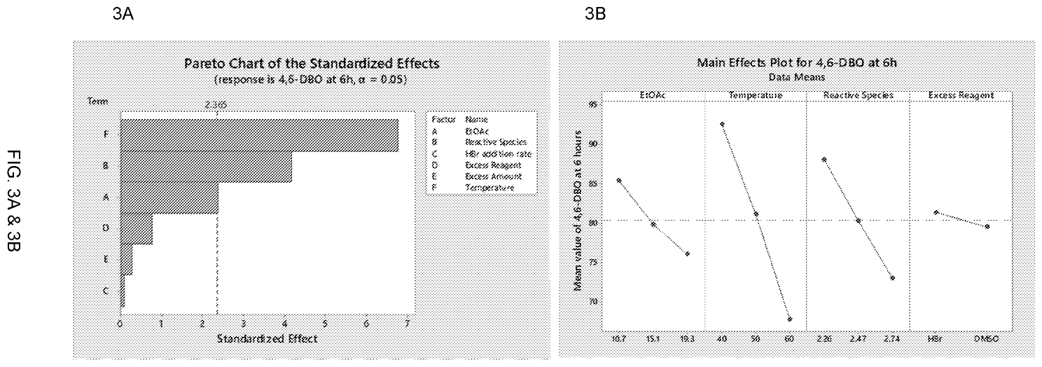

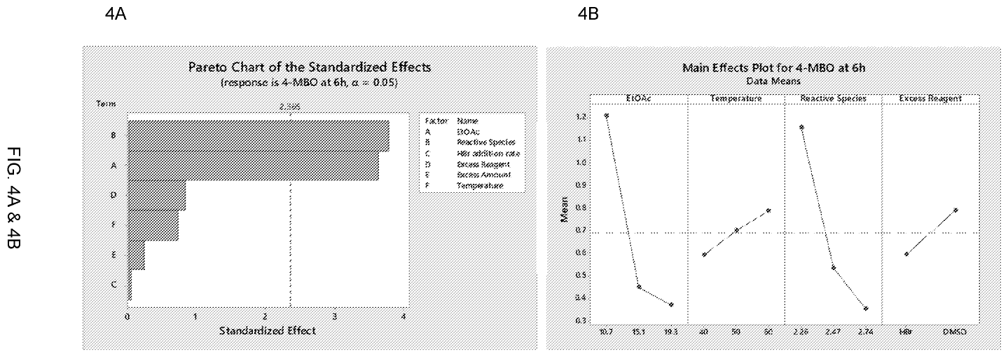

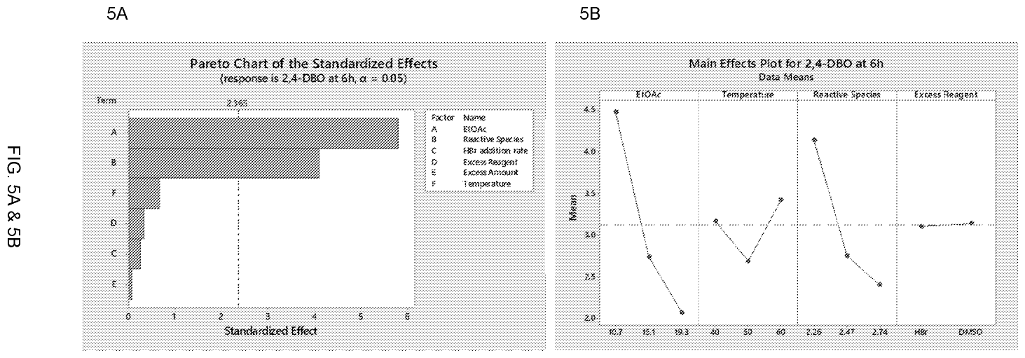

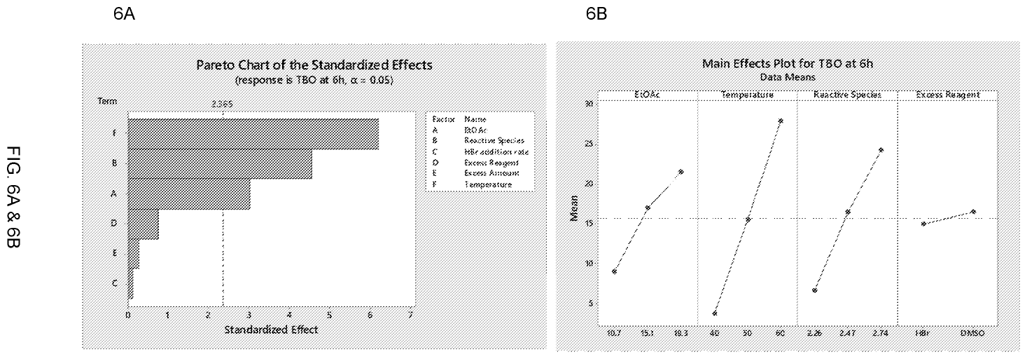

Results are shown in the form of a Pareto chart of standardized effects with a criterion of 2.365, and the main effect plots describing the various species formed under operating ranges in Table 10 and FIGS. 3A, 3B, 4A, 4B, 5A, 5B, 6A and 6B.

TABLE 10
Experimental Screen Design (13 experiments, 6 Factors)

| Factors | Units | Low | Center | High |
|---|---|---|---|---|
| Ethyl acetate | L/kg | 10.7 | 15.1 | 19.3 |
| Reactive Species | mol/mol | 2.26 | 2.47 | 2.74 |
| HBr addition rate | eq/min | 0.11 | 0.47 | 0.43 |
| Excess Reagent | DMSO or HBr | HBr or DMSO | | |
| Excess Amount | mol/mol | 0.00 | 0.05 | 0.10 |
| Temperature | ° C. | 40 | 50 | 60 |

### Example 11: Scale Up of Modified Reaction

The objective of this experiment was to investigate the feasibility of 15 g scale-up experiment under reaction conditions determined in Example 10. In this experiment, Olivetol (15 g, not corrected for water %) and solvent (16 vol.) were charged to a 300 mL reactor. To the solution was added DMSO (2.20 equiv.) and 48% HBr (2.26 equiv.) was charged in a controlled manner. The reaction was heated to 40° C. under agitation and aliquots were taken for HPLC analysis at 0.5, and 1 hour during HBr addition at 20° C.; sampled at 0.5, 1, 2, and 4 hours at 40° C. after HBr addition. At the end of 4 hours, the reaction mixture was cooled to 20° C. A buffer solution consisting of 18% NaOH in the amount of 2 Eq. added used to quench the bromination reaction and bring the solution to a final pH of 5.52. The solution appeared bi-phasic with no salt formation. The reaction mixture was distilled under vacuum to 5 L/kg. To the biphasic solution was then added 10 L/kg n-heptane and the reaction mixture was distilled under vacuum again to 5 L/kg. Another charge of heptane was added to the reaction mixture and distilled under vacuum to 10 L/kg. A slurry of solids was observed. The solution was cooled to 50° C. and water was added. The solids in the solution crystallized instantaneously and the mixture was aged (under agitation) for 1 hour. The solution was then cooled to 20° C. and filtered. Solids were isolated over filter paper. The solid cake was washed with 3 L/kg heptane and isolated solids were dried under vacuum at 45° C. overnight. HPLC results for dried DBO solids are shown in Table 11 and FIG. 2. The yield of dried DBO solids was found to be 89%.

TABLE 11
HPLC Peak Results

| | | Retention | | % | |
|---|---|---|---|---|---|
| | Species | time (min) | Area | Area | Height |
| 1 | 4-Bromo Olivetol | 5.286 | 17454 | 0.34 | 2757 |
| 2 | 2,4-Dibromo-Olivetol | 7.054 | 11592 | 0.23 | 1445 |
| 3 | 4,6-Dibromo-Olivetol | 8.728 | 5065560 | 99.33 | 626639 |
| 4 | 2,4,6-Tribromo- | 10.673 | 5014 | 0.10 | 667 |
| | Olivetol | | | | |

### Example 13: Purity Profile

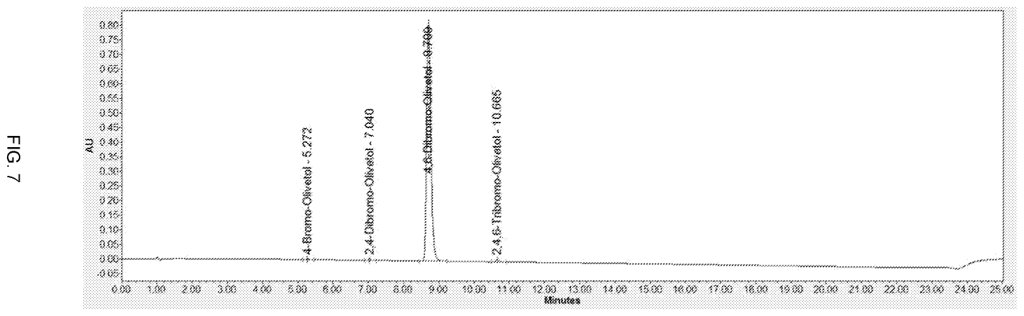

The objective of this experiment is to investigate final impurities in DBO solids in a 15 g scale-up experiment under reaction conditions favoring high TBO formation. In this experiment, Olivetol (15 g, not corrected for water %) and solvent (16 vol.) were charged to a 300 mL reactor. To the solution was added DMSO (2.35 equiv.) and 48% HBr (2.40 equiv.) was charged in a controlled manner. The reaction was heated to 46° C. under agitation and aliquots were taken for HPLC analysis at 0.5, and 1 hour during HBr addition at 20° C.; sampled at 0.5, 1, 2, and 4 hours at 46° C. after HBr addition. At the end of 4 hours, the reaction mixture was cooled to 20° C. A buffer solution consisting of 18% NaOH in the amount of 2 Eq. added used to quench the bromination reaction and bring the solution to a final pH in the range between 5-6. The solution appeared bi-phasic with no salt formation. The reaction mixture was distilled under vacuum to 5 L/kg. To the biphasic solution was then added 10 L/kg n-heptane and the reaction mixture was distilled under vacuum again to 5 L/kg. Another charge of heptane was added to the reaction mixture and distilled under vacuum to 10 L/kg. A slurry of solids was observed. The solution was cooled to 50° C. and water was added. The solids in the solution crystallized instantaneously and the mixture was aged (under agitation) for 1 hour. The solution was then cooled to 20° C. and filtered. Solids were isolated over filter paper. The solid cake was washed with 3 L/kg heptane and isolated solids were dried under vacuum at 45° C. overnight. HPLC results for dried DBO solids are shown in Table 12 and FIG. 7. The yield was found to be 90.6%.

TABLE 12
HPLC Peak Results

| | | Retention | | % | |
|---|---|---|---|---|---|
| | Species | time (min) | Area | Area | Height |
| 1 | 4-Bromo Olivetol | 5.272 | 13861 | 0.2 | 2102 |
| 2 | 2,4-Dibromo-Olivetol | 7.040 | 12150 | 0.18 | 1606 |
| 3 | 4,6-Dibromo-Olivetol | 8.709 | 6723327 | 98.72 | 814086 |
| 4 | 2,4,6-Tribromo- | 10.665 | 60916 | 0.89 | 7885 |
| | Olivetol | | | | |

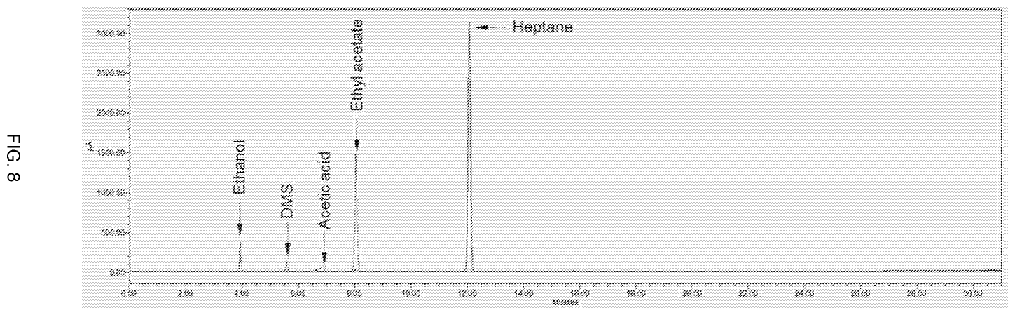

GC analysis of process stream before and after solvent swap (ethyl acetate swapped with heptane) distillation showed evidence of ethanol and acetic acid, by-products of ethyl acetate hydrolysis. DMS is also be present as the by-product of DMSO. An example of GC analysis is shown in FIG. 8.

Efforts have been made to ensure accuracy with respect to numbers used (e.g. amounts, temperature, etc.) but some experimental errors and deviations should be accounted for.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, N.Y.; and Janeway, C., Travers, P., Walport, M., Shlomchik (2001) Immunobiology, 5th Ed., Garland Publishing, New York.

The disclosures of all cited references including publications, patents, and patent applications are expressly incorporated herein by reference in their entirety.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller ranges is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation. One skilled in the art will recognize many methods and materials similar or equivalent to those described herein, which could be used in the practicing the subject matter described herein. The present disclosure is in no way limited to just the methods and materials described.

1. A method of preparing a compound of Formula I: 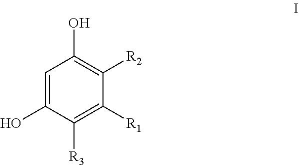
wherein,
R₁ is a branched or straight chain C₁₋₁₂ alkyl; and
R₂ and R₃ are each independently selected from a group consisting of halogen, —C(O)O—C₁₋₆ alkyl, and hydrogen, wherein at least one of R₂ and R₃ is halogen, the method comprising:
contacting a compound of Formula I′ having a structure: 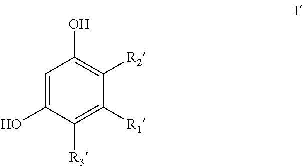
wherein, R_{1′} is a branched or straight chain C₁₋₁₂ alkyl; and
R_{2′} and R_{3′} are each independently selected from the group consisting of halogen, —C(O)O—C₁₋₆ alkyl, and hydrogen, wherein at least one of R_{2′} and R_{3′} is hydrogen;
with HX, wherein X is a halide, in the presence of an organic sulfoxide
wherein, the contacting is at a temperature from about 0° C. to about 100° C.; and
wherein, the compound of Formula I is prepared.

2. The method of claim 1, wherein HX is selected from HBr, HCl, HI, and HF.

3.-4. (canceled)

5. The method of claim 1, wherein R₁ and R_{1′} are the same and each is selected from the group consisting of straight or branched methyl, ethyl, propyl, isopropyl, n-butyl, sec-butyl, isobutyl, t-butyl, pentyl, hexyl, heptyl, octyl, nonyl, decyl, undecyl, and dodecyl.

6. (canceled)

7. The method of claim 1, wherein R₁ and R_{1′} are the same and each is selected from the group consisting of a branched chain C₁₋₁₂ alkyl having one, two, three, four, five, six, seven, eight, nine, ten, eleven, or twelve carbon atoms.

8. The method of claim 1, wherein the compound of Formula I is a compound having a structure: 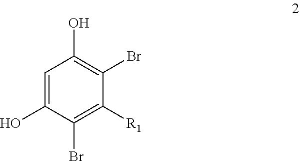
wherein R₁ and R_{1′} are the same.

9. The method of claim 8, wherein R₁ and R_{1′} are each selected from the group consisting of straight or branched methyl, ethyl, propyl, isopropyl, n-butyl, sec-butyl, isobutyl, t-butyl, pentyl, hexyl, heptyl, octyl, nonyl, decyl, undecyl, and dodecyl.

10.-11. (canceled)

12. The method of claim 9, wherein the compound of Formula I is a compound having a structure: 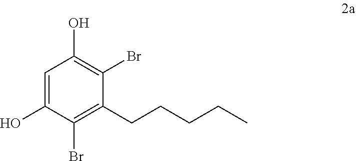
wherein said compound has a purity above about 93A % by HPLC.

13.-14. (canceled)

15. The method of claim 1, wherein the compound of Formula I′ is selectively di-halogenated in the 4 and 6 positions.

16. The method of claim 15, wherein the 4,6-di-halogenated compound of Formula I, wherein each of R₁ and R₂ is halogen is prepared at a ratio of from about 25:1 to about 34:1 relative to the 2,4-dihalogenated impurity compound.

17. The method of claim 1, wherein prior to said contacting, the compound of Formula I′ is contacted with a first solvent to form a mixture.

18. (canceled)

19. The method of claim 17, wherein the solvent is selected from the group consisting of ethyl acetate, isopropyl acetate, acetonitrile, and acetone.

20. (canceled)

21. The method of claim 17, wherein the solvent is present from about 9.7 vol to about 16.1 vol.

22. The method of claim 1, wherein the organic sulfoxide is present in an amount of about 2.0 equiv to about 3.0 equiv.

23. The method of claim 22, wherein the HX is present in an amount from about 2.0 equiv to about 3.0 equiv.

24.-26. (canceled)

27. A method of preparing a compound of Formula I: 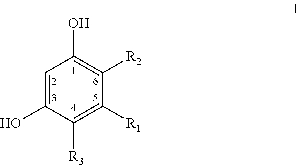
wherein,
R₁ is a branched or straight chain C₁₋₁₂ alkyl; and
R₂ and R₃ are each halogen,
the method comprising:
selectively halogenating at the 4- and 6-positions by contacting a compound of Formula I′ having a structure: 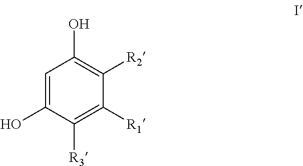
wherein, R_{1′} is a branched or straight C₁₋₁₂ alkyl; and
R_{2′} and R_{3′} are each hydrogen;
with a first solvent to form a mixture,
contacting the mixture with HX, wherein X is a halide, in the presence of an organic sulfoxide;
wherein, the contacting is at a temperature from about 0° C. to about 100° C.; and
wherein, the compound of Formula I is prepared.

28. The method of claim 27, wherein the compound of Formula I is present at a ratio of at least 10:1 relative to a mono-halogenated, tri-halogenated or 2,4-dihalogenated compound.

29.-30. (canceled)

31. The method of claim 27, wherein the halide is Br and HX is HBr.

32.-35. (canceled)

36. The method of claim 27, wherein R_{1′} is propyl or pentyl, and the compound of Formula I is selected from the group consisting of:

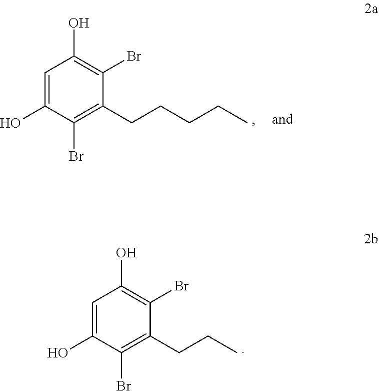

37.-39. (canceled)

40. A composition comprising 4,6-DBO; 4-MBO; 2,4-DBO and TBO in amounts of about 94%, about 3%, about 1%, and about 1%, respectively.

41. The method of claim 1 or 27, wherein said organic sulfoxide is of the general formula: 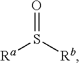
wherein, Ra and Rb are each independently selected from the group consisting of benzyl, phenyl, alkyl, aryl and allyl.

42.-43. (canceled)
